(19) United States
(12) Patent Application Publication
(10) Pub. No.: US 2023/0309810 A1
(43) Pub. Date: Oct. 5, 2023

(54) MEDICAL FLUID TEMPERATURE MANAGEMENT SYSTEM

(71) Applicant: Keymed (Medical & Industrial Equipment) Limited, Southend-on-Sea (GB)

(72) Inventor: Gareth SYKES, Southend-on-Sea (GB)

(73) Assignee: Keymed (Medical & Industrial Equipment) Limited, Southend-on-Sea (GB)

(21) Appl. No.: 18/113,858

(22) Filed: Feb. 24, 2023

Related U.S. Application Data
(60) Provisional application No. 63/325,192, filed on Mar. 30, 2022.

(51) Int. Cl. A61B 1/12; A61B 1/015

(52) U.S. Cl. CPC A61B 1/128; A61B 1/015

(57) ABSTRACT

A medical fluid temperature management system for a medical procedure including: a container containing fluid for supply to a medical instrument; a pump operable to withdraw fluid from the container and supply fluid to the medical instrument; a heater located externally to the container and configured to heat the fluid in the container; a first sensor to detect an ambient temperature; a second sensor to detect a temperature of the heater; and a processor in communication with the first and second sensors and the processor adjusting operating parameter(s) of the heater based on a difference between a detected ambient temperature and temperature of the heater, and based on a relationship between the temperature of the heater and a temperature of the fluid in the container and a relationship between the ambient temperature and the temperature of the heater, to maintain the temperature of the fluid within a predetermined temperature range.

### CROSS-REFERENCE TO RELATED APPLICATION

The present application is based upon and claims the benefit of priority from U.S. Provisional Application No. 63/325,192, filed on Mar. 30, 2022, the entire contents of which is incorporated herein by reference.

### BACKGROUND

### Field

The present disclosure relates generally to a fluid temperature management system for medical procedures such as endoscopies. In particular, the disclosure relates to a system and method for controlling the temperature of irrigation fluid used in a medical procedure.

### Prior Art

Endoscopic procedures involve the insertion of a medical instrument into a patient's body to observe an internal organ or tissue in detail for the purposes of imaging, investigation, diagnosis or surgery/treatment. Such endoscopic procedures often involve supplying irrigation fluid into the patient's body to irrigate and clean the site being observed. For example, in gastrointestinal (GI) procedures, irrigation is helpful to wash gastric and colonic mucosa to remove blood, faeces or other organic matter. Irrigation fluid is supplied by a pump into a channel in the endoscope itself.

The irrigation fluid may be heated. This makes the procedure more comfortable for the patient and reduces the chance of cold liquid inducing muscular spasms. A bottle containing irrigation fluid may be warmed before connection to a pump and the endoscope. It is also known to place a bottle of irrigation fluid on a stand or holder which incorporates an electrically powered heating plate. Although this aims to maintain the temperature of the fluid in the bottle, in practice it is found to be less effective as heat losses from the bottle are very great and there is no adjustment to varying ambient temperature. There is therefore a need for an improved fluid management system and method which can reliably provide heated fluid to a medical instrument.

### SUMMARY

Accordingly, a medical fluid temperature management system for a medical procedure is provided. The medical fluid temperature management system comprising a container containing fluid for supply to a medical instrument, a pump operable to withdraw fluid from the container and supply it to a medical instrument, a heating device located external to the container and configured to heat the fluid in the container, and a control system configured to regulate the temperature of the fluid in the container to within a predetermined temperature range, wherein the control system comprises a first temperature sensor configured to detect the ambient temperature, a second temperature sensor configured to detect the temperature of the heating device, and a processor in communication with the first temperature sensor and with the second temperature sensor, the processor configured to adjust operating parameters of the heating device in response to the difference between the detected ambient temperature and the detected temperature of the heating device, and in accordance with a first predetermined relationship between the temperature of the heating device and the temperature of fluid in the container, and in accordance with a second predetermined relationship between the ambient temperature and the temperature of the heating device, in order to maintain the temperature of fluid in the container within a desired temperature range.

The heating device may contact the base and/or a side surface of the container. The system may further comprise a support structure on which the container is removably received, and the heating device may be located in the support structure. The support structure may define an insulated enclosure for receiving the container. The enclosure may be shaped and dimensioned to form a close fit with the container. At least a part of the enclosure may be formed of a resilient material. The heating device may comprise an electrically heated plate or wire.

The first temperature sensor may be located externally of the pump. Alternatively, the first temperature sensor may be located internally of the pump and detects an internal ambient temperature within the pump, and the second predetermined relationship may be adjusted in accordance with a third predetermined relationship between the internal ambient temperature and an external ambient temperature external to the pump.

The system may be configured to maintain the temperature of the fluid in the container to 37±3° C.

Also provided is a method of controlling the temperature of fluid used in a medical procedure. The method comprising providing a container containing fluid for supply to a medical instrument, a pump operable to withdraw fluid from the container and supply it to the medical instrument, a heating device located external to the container and configured to heat the fluid in the container, and a control system comprising a first temperature sensor configured to detect the ambient temperature, a second temperature sensor configured to detect the temperature of the heating device, and a processor in communication with the first temperature sensor and with the second temperature sensor; the method further comprising: measuring the ambient temperature with the first temperature sensor, measuring the temperature of the heating device with the second temperature sensor and using the processor to adjust operating parameters of the heating device in response to the difference between the detected ambient temperature and the detected temperature of the heating device, and in accordance with a first predetermined relationship between the temperature of the heating device and the temperature of fluid in the container, and in accordance with a second predetermined relationship between the ambient temperature and the temperature of the heating device, in order to maintain the temperature of fluid in the container within a desired temperature range.

The control system may be configured to maintain the temperature of the fluid in the container at 37±3° C.

The method may further comprise placing the container in an insulated enclosure to reduce heat loss from the container, and adjusting the first predetermined relationship to account for the reduced heat loss.

Adjusting operating parameters of the heating device may comprise at least one of adjusting a power supply to the heating device and switching the heating device on and off for certain intervals.

The first temperature sensor may be located internally of the pump and detect an internal ambient temperature within the pump, and the method may further comprise adjusting the second predetermined relationship in accordance with a third predetermined relationship between the internal ambient temperature and an external ambient temperature external to the pump.

### BRIEF DESCRIPTION OF THE DRAWINGS

The embodiments will now be described in detail, by way of example only, with reference to the accompanying drawings in which:

FIG. 1 illustrates a schematic front view of a fluid management system;

FIG. 2 illustrates a schematic side view of a pump with an alternative form of bottle holder;

FIG. 3 illustrates a schematic perspective view of another form of bottle holder;

FIG. 4 illustrates a schematic cross section of the bottle holder of FIG. 3; and

FIG. 5 illustrates a schematic view of another alternative form of bottle holder.

### DETAILED DESCRIPTION

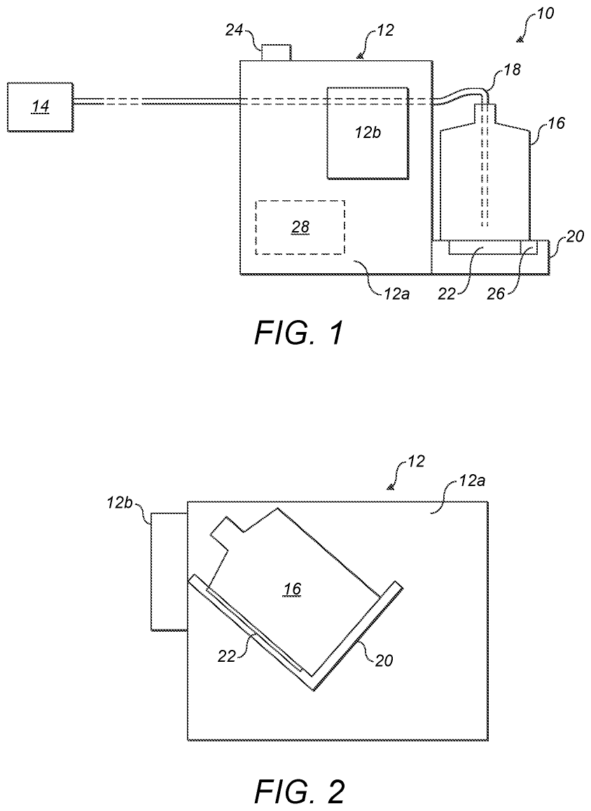

Referring first to FIG. 1, a fluid temperature management system 10 is shown. The fluid temperature management system 10 comprises a pump 12 which is operable to supply fluid to a medical instrument 14, for example an endoscope (which is illustrated only schematically). The fluid is supplied from a container 16, which may be a plastic bottle. The pump 12 may be a peristaltic pump, with a pump body 12a and a pump head 12b. In this case, fluid is withdrawn from the bottle 16 via flexible tubing 18, which passes through the pump head 12b and connects to the medical instrument 14. The bottle 16 is located on a holder 20, which may be fixed adjacent to the pump 12. The bottle 16 is filled with sterile fluid and the bottle 16 can be removed and replaced with another bottle when it is empty.

The holder 20 may comprise a flat support such as a tray or shelf on which the bottle 16 stands in an upright position as shown in FIG. 1. Alternatively, shown in FIG. 2, the holder 20 may be configured to support the bottle 16 in a titled position. In this example, the holder 20 is in the form of a substantially L-shaped bracket, secured to the side of the pump body 12a at an angle, which may be about 45°. The actual configuration of the holder 20 and the angle may vary but it will be apparent that generally this form of holder 20 supports the bottle 16 leaning on its side. In such configuration, the bottle 16 can tilt forward, towards the front of the pump 12 and thus towards a user, in order to facilitate attaching tubing 18 to the bottle 16 and to the pump head 12b.

The holder 20 further comprises a heating device 22 which is used to heat the fluid in the bottle 16. For example, the heating device 22 may be an electrically heated conductive plate, or other heating elements, such as wires. The heating device 22 may be set into the base of the holder 20 as shown in FIG. 1, so that it contacts the base of the bottle 16. Alternatively, or in addition, the heating device 22 may be set into the side of the holder 20 so as to contact the side of a bottle 16, for example as shown in FIG. 2. The bottle 16 stands on and/or leans against the heating device 22 so that as much of the area of the heating device 22 as possible contacts the bottle 16 for optimum heat transfer.

The system 10 further comprises a temperature control system to maintain the temperature of fluid within the bottle 16 to within a predetermined range which is suitable for administration to a patient. If the fluid is too hot or too cold it may cause discomfort or pain to the patient, or even risk internal injury. As normal body temperature is approximately 37° C., the predetermined temperature range can be a few degrees either side of this, for example it may be 37±3° C.

The ambient temperature, that is the air temperature of the surroundings in which the fluid management system 10 is to be used, is usually variable across a much larger range. For example, in different environments, the ambient temperature might be in the range of about 15° C. up to about 40° C. Therefore, the temperature of fluid in the bottle 16 must be controlled within a much tighter temperature range than the typical ambient temperature range.

It is not possible to directly measure the temperature of the fluid in the bottle 16 because the fluid must be maintained in a sterile condition for administration to a patient and therefore a temperature probe or sensor cannot be brought into direct contact with the fluid. Temperature measurement on the exterior surface of the bottle 16 is normally not sufficiently accurate because the bottles 16 are typically formed of plastic which has some thermal insulating effect, and this will vary with the specific material used and thickness of the wall of the bottle 16. Therefore, the temperature of the fluid in the bottle 16 must be regulated without that temperature being measured directly. The ambient temperature and temperature of the heating device 22 itself can be measured and the temperature information can be used to control the heating device 22, in order to regulate the temperature of the fluid in the bottle 16.

A relationship R1 between the temperature of the heating device 22 and the temperature of the fluid in a particular type of bottle 16 is established in advance. In other words, through prior testing, the temperature of the heating device 22 itself and the resultant temperature achieved for a particular fluid in a known type of bottle 16 placed in contact with the heating device 22 is mapped. The relationship R1 may depend on a number of factors including the material, wall thicknesses and shape of the bottle, the volume and type of fluid in the bottle, the contact area between the heating device 22 and the bottle 16, the power supply to and output from the heating device 22 and so on.

Thus, for a given type of bottle 16 and fluid, and a given type of heating device 22, a relationship R1 is determined in advance and which defines the operating parameters of the heating device 22 which will be required in order for the heating device 22 to heat a given bottle 16 of fluid to a desired temperature.

However, there will be heat losses from the bottle 16 over time. This heat loss will vary with the ambient temperature. The ambient temperature is the air temperature of the surroundings in which the system 10 is being used and the medical procedure is being carried out. Heat loss from the bottle 16 is not a linear relationship with ambient temperature. Therefore, the effect of ambient temperature on the rate of heat loss from the bottle 16 can be established in advance and thus what temperature will be required at the heating device 22 to maintain the fluid temperature in the desired range.

A relationship R2 between the ambient temperature and the temperature of the heating device 22 is also established in advance by appropriate prior testing. The relationship R2 concerns the effect of ambient temperature on the output temperature of the heating device 22 which will be required to maintain the fluid temperature in the desired range. When the ambient temperature is higher, the heating device 22 temperature can be lower to achieve the desired fluid temperature and conversely, when the ambient temperature is lower, the heating device temperature must be higher to achieve the desired fluid temperature.

By way of example only, when the ambient temperature is +33° C. or higher, the heating device 22 may not be needed at all and can be switched off. When the ambient temperature is between 28 to 32° C., the heating device 22 may be run at between 43 to 45° C. When the ambient temperature is between 22 to 27° C. the heating device 22 may be run at between 48 to 50° C. When the ambient temperature is 21° C. or lower, the heating device 22 may be run at between 52 to 54° C.

Depending on the ambient temperature, the power required to bring the heating device 22 to a desired temperature will vary. Thus, the relationship R2 can be used to determine the operating parameters of the heating device 22 which will be required in order to obtain a desired output temperature of the heating device 22 in different ambient conditions. The relationship R2 may depend on a number of factors including the power supply to the heating device 22, the surface area of the heating device 22 and so on.

As noted above, the relationships R1 and R2 are determined in advance. During use of the fluid temperature management system 10, the ambient temperature is monitored by a first temperature sensor 24. This may be mounted on the pump 12 as shown schematically in FIG. 1, or on other nearby equipment. Alternatively, the first temperature sensor 24 may be located within the body of the pump 12. In this case, the internal ambient temperature within the pump 12 may be slightly higher than the external ambient temperature, due to operation of the pump 12. The difference between the external and internal ambient temperatures may therefore also be mapped in advance through prior testing to establish a third relationship R3, and appropriate correction made to the relationship R2 to take account of the third relationship R3.

In addition, the temperature of the heating device 22 is measured by a second temperature sensor 26, shown schematically in FIG. 1. Typically, the temperature is measured at the surface of the heating device 22 which contacts the bottle 16 in use.

A processor 28 (such as a CPU, computer or circuit), which may be located within the pump 12 as indicated schematically in FIG. 1, or elsewhere, receives information from the first and second temperature sensors 24, 26 regarding the ambient temperature and the temperature of the heating device 22. The temperature information may be transmitted from the sensors 24, 26 to the processor 28 wirelessly, or a wired connection may be provided. Based upon the temperature information and the predetermined relationships R1 and R2, which can pre-programmed into the processor 28, can be available from a separate storage device having one or more databases reflecting the predetermined relationships or can be available remotely though an internet or other wireless connection, the processor 28 is configured to control operating parameters of the heating device 22 in order to alter the temperature of the heating device 22 so that it will heat fluid in the bottle 16 to within the desired temperature range. The heating device 22 may be adjusted for example by increasing or decreasing the power supply (not shown) and/or by switching the heating device 22 on and off for certain intervals. Furthermore, the processor 28 may be used to control the pump, such as altering the speed (e.g., flow rate) of the pump.

As noted above, when the ambient temperature is high, the heating device 22 needs to be at a lower temperature in order to maintain a desired temperature of the fluid in the bottle 16. On the other hand, in very cool ambient conditions, the heating device 22 will need to be hotter to achieve the desired temperature range for the fluid in the bottle 16. Thus, according to the first predetermined relationship R1 between the temperature of the heating device 22 and the temperature of fluid in the bottle 16, the second predetermined relationship R2 between the ambient temperature and the temperature of the heating device 22, and the actual measured difference between the ambient temperature and the temperature of the heating device 22, the system 10 is configured to control the heating device 22 to maintain the temperature of the fluid in the bottle 16 to within a desired temperature range. Such a system can ensure patient comfort and safety and provide for efficient use of power for the heating device 22.

Various other correction factors may also be calculated to maximize the efficiency of the system. For example, the change in volume of fluid as it is withdrawn from the bottle 16 may have an effect on the relationships R1 and R2, which can be established in prior testing.

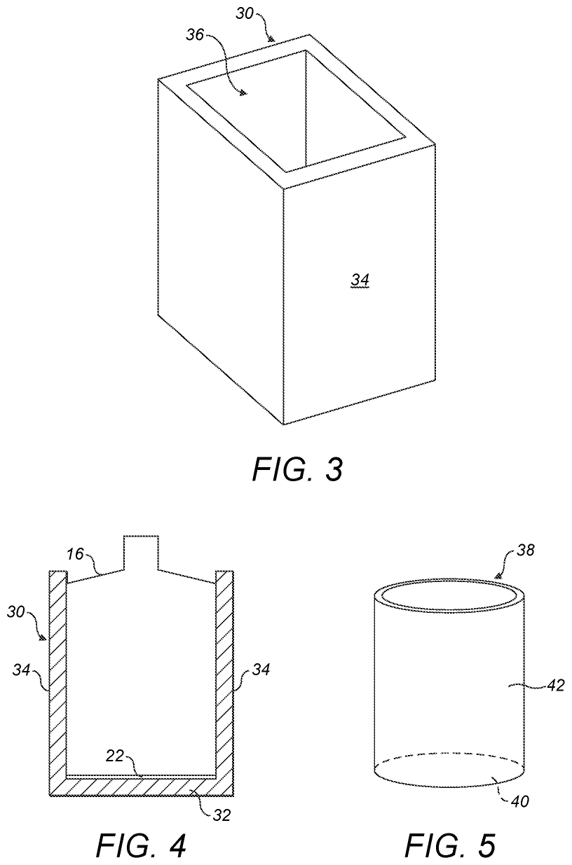

Another development is illustrated in FIGS. 3-5. These figures show alternative forms of holder 30, 38 which comprise an insulated enclosure for receiving the bottle 16. The insulated enclosure reduces heat losses from the heating device 22 and from the bottle 16 and therefore keeps the temperature of the fluid in the bottle 16 more stable, minimizing power consumption of the heating device 22.

For example, instead of a simple tray on which the bottle stands or leans, as in FIGS. 1 and 2, in FIGS. 3 and 4, the holder 30 comprises a base 32 and side walls 34 which together define an enclosure 36 into which the bottle 16 may be placed. The enclosure 36 is sufficiently deep to receive substantially the entire body of the bottle 16 with just the neck protruding to allow for attachment of the tubing 18 leading to the pump 12 and the medical instrument 14. The enclosure 36 is shaped to fit a given configuration of the bottle 16. For example, in this case the bottle is substantially square or rectangular in horizontal cross section and thus the enclosure 36 will be also substantially square or rectangular in horizontal cross section, as seen in FIG. 3, and dimensioned to receive the bottle 16 with a very close fit so there is good contact between the bottle 16 and the base and side walls of the holder 30 and minimal air gaps, as seen in FIG. 4. For a bottle 16 with a substantially circular horizontal cross section, a cylindrical enclosure may be provided, as shown schematically by the holder 38 of FIG. 5.

The base and side walls of the holder 30, 38 may comprise thermally insulating material. Alternatively, or in addition, the base and side walls of the holder 30, 38 may comprise a double-walled structure with an evacuated cavity therebetween. The holder 30, 38 may also be provided with a reflective coating, such as on the interior surface, to prevent heat losses by radiation.

A heating device 22 may be provided in the base of the holder 30, 38. Alternatively, or in addition, the heating device 22 may be provided in one or more side walls of the enclosure so that there is a greater contact area between the heating device 22 and the bottle 16.

The holder 30, 38 may be a substantially rigid structure. Alternatively, part of the holder may be formed of a resilient material. For example, with a cylindrical holder 38 as shown in FIG. 5, the cylindrical side wall may be in the form of a resilient sleeve 42 secured to a base 40. The resilient sleeve 42 may be dimensioned to be slightly smaller than the bottle 16 so that it must be stretched to fit the bottle 16, to maintain a tight fit around the bottle 16. Although not shown in FIG. 5, the base 40 and/or the sleeve 42 may incorporate a heating device 22. This may be a rigid plate set into the base 40 and/or there may be wires within the sleeve 46, for example extending vertically to allow for circumferential stretching of the sleeve around a bottle 16.

The insulation will reduce the heat losses from the bottle 16. Therefore, the relationships R1 and R2 may need to be adjusted or correction factors applied to suit a system employing an insulated holder.

Use of an insulated holder in this way improves the efficiency of the system. Less power is required initially to heat the fluid in the bottle 16 to a desired temperature, and to maintain the temperature of the fluid within the desired range thereafter. Since the fluid temperature in the bottle 16 is maintained with fewer losses, this simplifies the control required and minimises the amount of adjustment needed to the heating device 22. While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A medical fluid temperature management system for a medical procedure, the medical fluid temperature management system comprising:
a container configured to contain fluid for supply to a medical instrument;
a pump operable to withdraw the fluid from the container and supply the fluid to the medical instrument;
a heater located externally to the container and configured to heat the fluid in the container;
a first temperature sensor configured to detect an ambient temperature;
a second temperature sensor configured to detect a temperature of the heater; and
a processor comprising hardware, the processor being in communication with the first temperature sensor and with the second temperature sensor and the processor being configured to adjust one or more operating parameters of the heater in response to a difference between a detected ambient temperature and a detected temperature of the heater, and in accordance with a first predetermined relationship between the detected temperature of the heater and a temperature of the fluid in the container, and in accordance with a second predetermined relationship between the detected ambient temperature and the detected temperature of the heater, in order to maintain the temperature of the fluid in the container within a predetermined temperature range.

2. The medical fluid temperature management system of claim 1, wherein the heater is configured to contact one or more of a base and a side surface of the container.

3. The medical fluid temperature management system of claim 1, further comprising a support on which the container is removably received, wherein the heater is located in the support.

4. The medical fluid temperature management system of claim 3, wherein the support defines an insulated enclosure for receiving the container.

5. The medical fluid temperature management system of claim 4, wherein the enclosure is shaped and dimensioned to form a close fit with the container.

6. The medical fluid temperature management system of claim 5, wherein at least a part of the enclosure is formed of a resilient material.

7. The medical fluid temperature management system of claim 1, wherein the heater comprises one or more of an electrically heated plate and wire.

8. The medical fluid temperature management system of claim 1, wherein the first temperature sensor is located externally of the pump.

9. The medical fluid temperature management system of claim 1, wherein the first temperature sensor is located internally of the pump and detects the ambient temperature within the pump, and the second predetermined relationship is adjusted in accordance with a third predetermined relationship between the detected ambient temperature within the pump and an ambient temperature external to the pump.

10. The medical fluid temperature management system of claim 1, wherein the predetermined temperature range is 37° C.±3° C.

11. A method of controlling the temperature of fluid used in a medical procedure, the method comprising:
using a first temperature sensor, measuring an ambient temperature;
using a second temperature sensor, measuring a temperature of a heater used to heat the fluid in a container; and
using a processor, adjusting one or more operating parameters of the heater in response to a difference between a detected ambient temperature and a detected temperature of the heater, and in accordance with a first predetermined relationship between the detected temperature of the heater and a temperature of the fluid in the container, and in accordance with a second predetermined relationship between the detected ambient temperature and the detected temperature of the heater, in order to maintain the temperature of the fluid in the container within a predetermined temperature range.

12. The method of claim 11, wherein the predetermined temperature range is 37° C.±3° C.

13. The method of claim 11, further comprising placing the container in an insulated enclosure to reduce heat loss from the container, wherein the adjusting further comprises adjusting the first predetermined relationship to account for the reduced heat loss.

14. The method of claim 11, wherein the one or more operating parameters of the heater comprise at least one of adjusting a power supply to the heater and switching the heater on and off for predetermined intervals.

15. The method of claim 11, wherein the first temperature sensor is located internally of the pump and detects the ambient temperature within the pump, and the method further comprises adjusting the second predetermined relationship in accordance with a third predetermined relationship between the detected ambient temperature within the pump and an ambient temperature external to the pump.

16. A control apparatus for controlling the temperature of fluid supplied to a medical instrument during in a medical procedure, the control apparatus comprising:
a processor comprising hardware, the processor being configured to:
receive a first output from a first temperature sensor indicating an ambient temperature;
receive a second output from a second temperature sensor indicating a temperature of the heater configured to heat the fluid contained in a container; and
adjust one or more operating parameters of the heater in response to a difference between the first output indicating the ambient temperature and the second output indicating the temperature of the heater, and in accordance with a first predetermined relationship between the second output indicating the temperature of the heater and a temperature of the fluid in the container, and in accordance with a second predetermined relationship between the first output indicating the ambient temperature and the second output indicating the temperature of the heater, in order to maintain the temperature of the fluid in the container within a predetermined temperature range.

17. The control apparatus of claim 16, wherein the predetermined temperature range is 37° C.±3° C.

18. The control apparatus of claim 16, wherein the processor is further configured to adjust the one or more operating parameters based on a reduced heat loss from the container resulting from the container being insulated.

19. The control apparatus of claim 16, wherein the one or more operating parameters of the heater comprise at least one of adjusting a power supply to the heater and switching the heater on and off for predetermined intervals.

20. The control apparatus of claim 16, wherein:
the first temperature sensor is located internally of the pump and the first output indicates the ambient temperature within the pump; and
the processor adjusts the second predetermined relationship in accordance with a third predetermined relationship between the first output indicating the ambient temperature within the pump and an ambient temperature external to the pump.
